(19) United States
(12) Patent Application Publication
(10) Pub. No.: US 2009/0259088 A1
(43) Pub. Date: Oct. 15, 2009

(54) Magnetic presser-expander device and treatment method using such device

(76) Inventors: Stefano Tassin, Seveso (IT); Massimo Donati, Seveso (IT); Paolo Pedicino, Seveso (IT)

Correspondence Address: Modiano & Associati, Via Meravigli 16, Milan, 20123, IT

(21) Appl. No.: 12/311,369

(22) PCT Filed: Oct. 10, 2007
(86) PCT No.: PCT/IB2007/003014

§ 371 Date: Mar. 27, 2009

(30) Foreign Application Priority Data
Oct. 12, 2006 (IT) MI2006A001955

(51) Int. Cl. A61N 2/08

(52) U.S. Cl. 600/15

(57) ABSTRACT

A magnetic presser-expander device for biological structures, constituted by two or more rare-earth magnets and means for applying said magnets to the skin, and methods for treating an alteration of an osteoarticular and/or musculoskeletal structure, a hematoma, an edema or an articular effusion by means of the presser-expander device.

The present invention relates to a magnetic presser-expander device for biological structures and to methods for treating alterations affecting the osteoarticular and/or musculoskeletal system and for the treatment of hematomas, edemas or articular effusions.

### BACKGROUND OF THE INVENTION

It is known that even small anatomical alterations of articular heads (dysplasias) entail changes to the distribution of the normal load and the cascade activation of “compensation” mechanisms by the musculoskeletal system, in order to ensure correct posture in the affected individual. For example, the presence of a valgus knee in a patient necessarily causes the onset of a varus ankle in order to keep the foot in a straight position. Such musculoskeletal compensation phenomena in the long run are responsible for a wide range of painful conditions in the affected individual, since alterations to the articular load cause inflammatory conditions with application of calcific tissue and excessive cartilage wear: the typical condition of arthrosis.

There is, therefore, the need to develop devices and methods which are capable of correcting osteoarticular and/or musculoskeletal alterations as early as possible.

The aim of the present invention is to provide a device which is capable of correcting osteoarticular and/or musculoskeletal alterations in a patient.

Within this aim, an object of the invention is to provide a method for treating osteoarticular and/or musculoskeletal alterations in a patient.

Another object of the invention is to provide a method for the treatment of edemas, hematomas and articular effusions in a patient.

Another object of the present invention is to provide a method for reducing pain and degenerative phenomena affecting the cartilages of articulations in patients affected by osteoarticular and/or musculoskeletal alterations.

### SUMMARY OF THE INVENTION

This aim and these and other objects, which will become better apparent hereinafter, are achieved by a magnetic presser-expander device for biological structures, constituted by two or more rare-earth magnets and by means for applying said magnets to a patient.

The aim and objects of the invention are also achieved by a method for treating an alteration of an osteoarticular and/or musculoskeletal structure in a patient by applying to the patient the device according to the invention, comprising the steps of:
(a) applying the magnets at the level of the osteoarticular and/or musculoskeletal structure affected by the alteration in a North-North position, so as to generate a repulsive magnetic field;
(b) applying the magnets at the level of the osteoarticular and/or musculoskeletal structure affected by the alteration in a North-South position, so as to generate an attractive magnetic field.

The intended aim and objects are also achieved by a method for “spacing” the articular heads in a patient affected by cartilage wear or by phlogosis of the articular rimae/capsules, of inflamed soft tissues by applying to the patient the device according to the invention, and comprising the step of:
a) applying the magnets at the level of the cartilage affected by wear or of the articular rimae/capsules or of the tissues affected by phlogosis in a North-North position, so as to generate a repulsive magnetic field.

Moreover, the aim and objects of the invention are achieved by a method for treating a traumatic or inflammatory hematoma, an edema or an articular effusion in a patient by applying to the patient the device according to the invention and comprising the step of:
(a) applying the magnets at the level of the hematoma, edema or articulation affected by effusion in a North-North position, so as to generate a repulsive magnetic field.

Finally, the intended aim and objects are also achieved by a method for applying an anti-inflammatory, analgesic and decontracturing action for the smooth and skeletal muscles in a patient by applying to the patient at the level of the affected muscles the device according to the invention.

### BRIEF DESCRIPTION OF THE DRAWINGS

Further characteristics and advantages of the invention will become apparent in the following detailed description, which must be considered together with the accompanying drawings, wherein:

FIG. 1 illustrates the elements that constitute the device according to the invention, the rare-earth magnets and a means for applying the magnets to the patient (elastic strap);

FIG. 2a is a view of the device according to the invention, in which the rare-earth magnets are joined one another with a curved bar which acts as a means for applying the magnets to the patient;

FIG. 2b is a view of the device according to the invention, in which the rare-earth magnets are joined one another with a straight bar;

FIGS. 3a and 3b are respectively front and back views of an application of the device according to the invention at the level of the articulation of the knee.

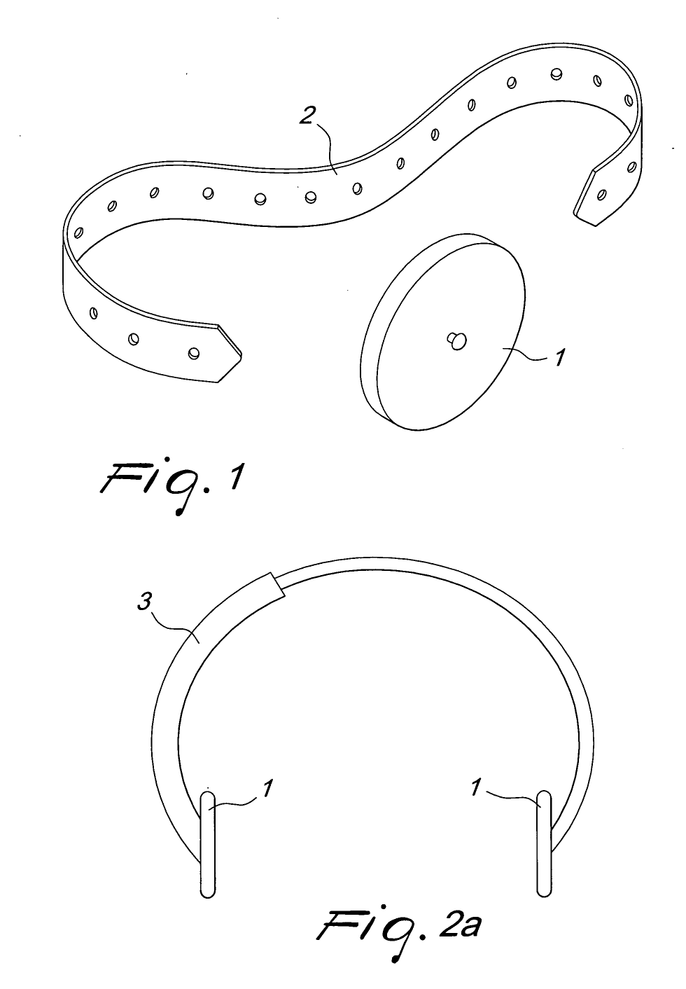

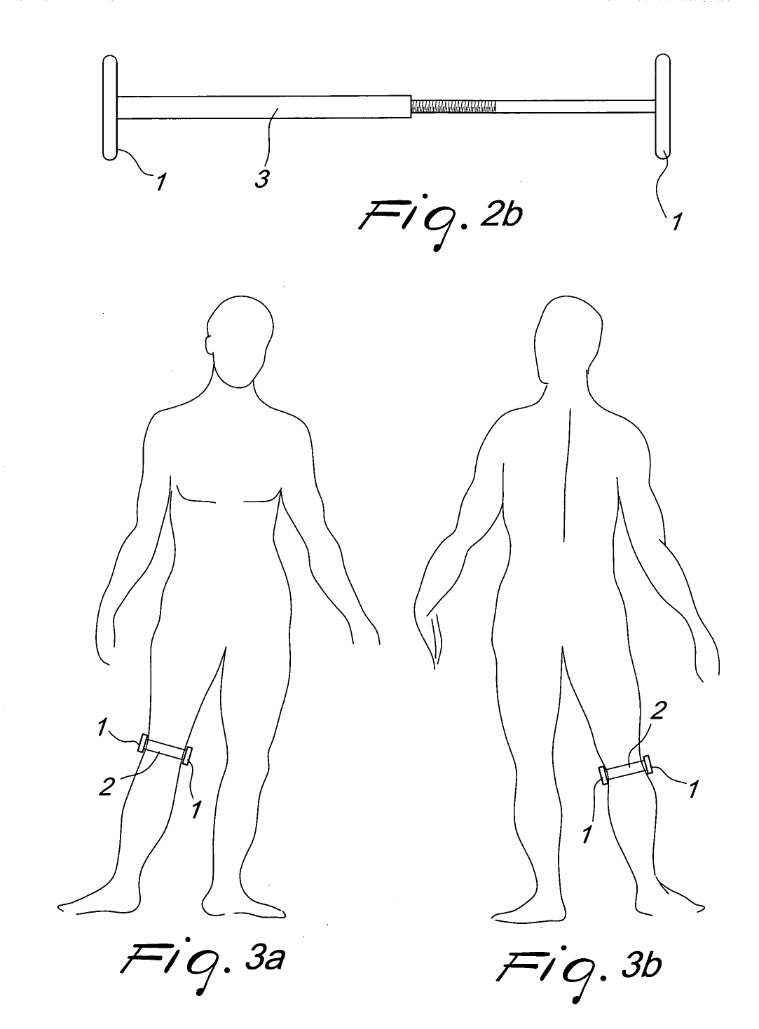

### DESCRIPTION OF THE PREFERRED EMBODIMENTS

The device according to the invention, hereinafter referenced as magnetic presser-expander, is based on the use in the biological field of the interaction between magnetic fields generated by two or more rare-earth magnets of which the device is composed. Rare-earth magnets are among the most powerful magnets known and are characterized by low weight and low costs. In particular, in an embodiment according to the invention, the two or more rare-earth magnets, designated by the reference numeral 1, can be magnets of the neodymium-iron-boron type. Generally, the strength of such magnets ranges from 150 to 15,000 gauss.

The rare-earth magnets of the device according to the invention can be characterized by different shape types (square, rectangular, circular, or annular), all of which are suitable for the purposes of the invention. In an embodiment of the device, the magnets can have thicknesses of no more than 30 mm.

The means suitable for applying to a patient the device according to the invention can be for example adhesive bandages, an adhesive fabric, an adhesive, elastic straps 2 or material with a curved bar 3, similar to earphones, or a straight adjustable bar 4. In particular, straps and materials shaped like a curved or straight bar which can be adjusted can be used to apply the device to articulations such as for example the knee, the wrist, the elbow, the shoulder or the tibia-tarsus. Moreover, the device according to the invention can also comprise two or more discs made of non-magnetized ferrous material (washers), to be matched up with the magnets during application to a patient in order to limit the power of the traction produced by the action of the magnetic fields.

A basic kit of the device according to the invention can comprise two higher-power magnets (from 5000 to 15,000 gauss), two lower-power magnets (from 150 to 5000 gauss), two washers made of non-magnetized ferrous material and one or more means for applying the magnets to the patient.

The device according to the invention uses the attractive or repulsive forces caused by the interaction of the magnetic fields generated by the rare-earth magnets to treat a biological structure of a patient to whom the device is applied. The forces produced by the interaction of the magnets act slowly and continuously, causing modifications to the biological structures that are affected by the generated pressures, with consequences, for example in the case of congenital or acquired dysplasias, on posture. The biological structures can be osteoarticular and/or musculoskeletal structures.

In particular, the present invention relates to a method for treating patients affected by osteoarticular and/or musculoskeletal alterations by means of the device described herein. The expression “osteoarticular and/or musculoskeletal alterations” is used to reference pathological situations affecting articular heads (dysplasias) and cartilage structures of articulations.

In the treatment of such alterations, the method provides for two steps: in a first step, the magnets 1 are applied to the patient at the osteoarticular and/or musculoskeletal structure affected by the alteration with a North-North polarity, in order to create a repulsive magnetic field at the level of the articular rima; in the second step, the magnets are arranged, again at the level of the structures affected by the alteration, with a North-South polarity, applying an attraction force between the diverted articular heads. The net result of this method is to restore the correct posture of the compromised osteoarticular and/or musculoskeletal structure. This structure can be selected for example from the group comprising the spine, the foot, the knee and the temporomandibular articulation.

A typical example of this application is the placement of the device according to the invention in disorders of the articulation of the knee: the application of the two magnets 1 in a North-North position (FIG. 3) to the articular rima produces a force field which tends to “space” the two articular heads, making the articulation work as if on a magnetic “bearing” which replaces the cartilage. The device is then applied with a North-South polarity, so as to use the direct attraction force between the two magnets to shift the altered structures. In the case of a knee affected by arthrosis, application of the magnets 1 with a North-South polarity can correct the imbalance of the articular heads caused by wear of the cartilage of one of the two parts.

Moreover, the invention also relates to a method for treating a traumatic or inflammatory hematoma, edema or articular effusion in a patient by applying to the patient the device according to the invention. In this case, the method provides for the application of the magnets 1 at the level of the hematoma, edema or articulation affected by effusion in a North-North position, so as to generate a repulsive magnetic field.

Moreover, the invention also relates to a method for applying an anti-inflammatory, analgesic and decontracturing action to the smooth and skeletal muscles of a patient affected by an osteoarticular and/or musculoskeletal alteration by applying to the patient, at the level of the affected muscles, the device described here. In this case, the action of the magnetic field generated by the device according to the invention reduces the pain caused by the pathological alteration and improves articular motility.

Finally, the invention also relates to a method for moving apart the articular heads in a patient affected by wear of the cartilages or by phlogosis of the articular rimae/capsules, of soft tissues which are inflamed or have hematomas by applying to the patient the device described herein, and comprising the step of applying the magnets 1 at the level of the cartilage affected by wear or of the articular rimae/capsules or of the tissues affected by phlogosis in a North-North position so as to generate a repulsive magnetic field.

The device according to the invention is applied for preset periods according to the disorder to be treated on precise points at the level of which the forces are to be applied; such forces, by acting continuously, induce persistent postural modifications. The force of the application of course depends on the strength of the magnets used.

It is important for the personnel/physician to be expert in human physiological and pathological osteoarticular complexity, and for the magnetic interaction to be never interrupted throughout the duration of the period of therapy, in order to avoid modifications of the force fields that have been created.

The term “patient”, as referenced in the present description, is to be understood generally as indicating a human being or an animal.

In practice it has been found that the device and the methods according to the invention fully achieve the intended aim, since the application of the device according to the invention allows to correct alterations affecting the osteoarticular and/or musculoskeletal structures and to reabsorb hematomas, edemas and articular effusions.

Moreover, the device and the methods according to the invention are capable of reducing pain and improving articular motility in patients affected by alterations of the osteoarticular and/or musculoskeletal structures.

The disclosures in Italian Patent Application no. MI2006A001955, from which this application claims priority, are incorporated herein by reference.

Where technical features mentioned in any claim are followed by reference signs, those reference signs have been included for the sole purpose of increasing the intelligibility of the claims and accordingly such reference signs do not have any limiting effect on the interpretation of each element identified by way of example by such reference signs. have been created.

The term “patient”, as referenced in the present description, is to be understood generally as indicating a human being or an animal.

In practice it has been found that the device and the methods according to the invention fully achieve the intended aim, since the application of the device according to the invention allows to correct alterations affecting the osteoarticular and/or musculoskeletal structures and to reabsorb hematomas, edemas and articular effusions.

Moreover, the device and the methods according to the invention are capable of reducing pain and improving articular motility in patients affected by alterations of the osteoarticular and/or musculoskeletal structures.

The disclosures in Italian Patent Application no. MI2006A001955, from which this application claims priority, are incorporated herein by reference.

What is claimed is:

1-11. (canceled)

12. A magnetic presser-expander device for biological structures, comprising two or more rare-earth magnets and means for applying said magnets to a patient.

13. The device according to claim 12, wherein the two or more rare-earth magnets are magnets of the neodymium-iron-boron type.

14. The device according to claim 12, wherein the two or more rare-earth magnets generate a magnetic field with a strength from 150 to 15,000 gauss.

15. The device according to claim 12, wherein the two or more rare-earth magnets have a thickness of no more than 30 mm.

16. The device according to claim 12, wherein the means for applying the magnets to the skin are selected from the group that comprises adhesive bandages, an adhesive fabric, an adhesive, elastic straps and material shaped like a curved or straight bar which can be adjusted.

17. The device according to claim 12, further comprising two or more discs made of ferrous material.

18. A method for treating an alteration of an osteoarticular and/or musculoskeletal structure in a patient by applying to the patient a device according to claim 12, comprising the steps of:
(a) applying the magnets at the level of the osteoarticular and/or musculoskeletal structure affected by the alteration in a North-North position, so as to generate a repulsive magnetic field;
(b) applying the magnets at the level of the osteoarticular and/or musculoskeletal structure affected by the alteration in a North-South position, so as to generate an attractive magnetic field.

19. The method according to claim 18, wherein the osteoarticular and/or musculoskeletal structure is selected from the group which comprises the spine, the foot, the knee and the temporomandibular articulation.

20. A method for spacing the articular heads in a patient affected by wear of the cartilages or by phlogosis of the articular rimae/capsules, of inflamed soft tissues or with hematomas by applying to the patient the device according to claim 12, comprising the step of:
a) applying the magnets at the level of the cartilage affected by wear or of the articular rimae/capsules or of the tissues affected by phlogosis in a North-North position, so as to generate a repulsive magnetic field.

21. A method for treating a traumatic or inflammatory hematoma, edema or articular effusion in a patient by applying to the patient the device according to claim 12, comprising the step of:
(a) applying the magnets at the level of the hematoma, edema or articulation affected by effusion in a North-North position, so as to generate a repulsive magnetic field.

22. A method for applying an anti-inflammatory, analgesic and decontracturing action for the smooth and skeletal muscles of a patient by applying to the patient at the level of the affected muscles the device according to claim 12.
